(19) United States
(12) Patent Application Publication
(10) Pub. No.: US 2001/0056231 A1
(43) Pub. Date: Dec. 27, 2001

(54) High resolution MRI imaging of brain functions

(76) Inventors: Andrzej Jesmanowicz, Wauwatosa, WI (US); James S. Hyde, Dousman, WI (US); Bharat B. Biswal, Milwaukee, WI (US)

Correspondence Address: Barry E. Sammons, Quarles & Brady, 411 East Wisconsin Avenue, Milwaukee, WI, 53202, US

(21) Appl. No.: 09/804,934

(22) Filed: Mar. 13, 2001

Related U.S. Application Data
(60) Provisional application No. 60/188,855, filed on Mar. 13, 2000.

(51) Int. Cl. A61B 5/055

(52) U.S. Cl. 600/410

(57) ABSTRACT

An EPI pulse sequence is performed by an NMR system which acquires images of the brain over a time interval during which the subject performs a function or is stimulated in a pattern. The voxel size of acquired images corresponds to the anatomy of cortical microcirculation structures which range from 1 to 2 mm along all three axes. A centric view order is employed and one-half of k-space is sampled to reduce scan time for each image.

### BACKGROUND OF THE INVENTION

The field of the invention is nuclear magnetic resonance imaging (MRI) methods and systems. More particularly, the invention relates to the production of brain function images (fMRI).

Any nucleus which possesses a magnetic moment attempts to align itself with the direction of the magnetic field in which it is located. In doing so, however, the nucleus processes around this direction at a characteristic angular frequency (Larmor frequency) which is dependent on the strength of the magnetic field and on the properties of the specific nuclear species (the magnetogyric constant γ of the nucleus). Nuclei which exhibit this phenomena are referred to herein as “spins”.

When a substance such as human tissue is subjected to a uniform magnetic field (polarizing field B₀), the individual magnetic moments of the spins in the tissue attempt to align with this polarizing field, but process about it in random order at their characteristic Larmor frequency. A net magnetic moment M_{z} is produced in the direction of the polarizing field, but the randomly oriented magnetic components in the perpendicular, or transverse, plane (x-y plane) cancel one another. If, however, the substance, or tissue, is subjected to a magnetic field (excitation field B₁) which is in the x-y plane and which is near the Larmor frequency, the net aligned moment, M_{z}, may be rotated, or “tipped” into the x-y plane to produce a net transverse magnetic moment Mₜ, which is rotating, or spinning, in the x-y plane at the Larmor frequency. The practical value of this phenomenon resides in the signal which is emitted by the excited spins after the excitation signal B₁ is terminated. There are wide variety of measurement sequences in which this nuclear magnetic resonance (“NMR”) phenomena is exploited.

When utilizing NMR to produce images, a technique is employed to obtain NMR signals from specific locations in the subject. Typically, the region which is to be imaged (region of interest) is scanned by a sequence of NMR measurement cycles which vary according to the particular localization method being used. The resulting set of received NMR signals are digitized and processed by reconstruction techniques. To perform such a scan, it is, of course, necessary to elicit NMR signals from specific locations in the subject. This is accomplished by employing magnetic fields (Gₓ, G_{y}, and G_{z}) which have the same direction as the polarizing field B₀, but which have a gradient along the respective x, y and z axes. By controlling the strength of these gradients during each NMR cycle, the spatial distribution of spin excitation can be controlled and the location of the resulting NMR signals can be identified.

The imaging of brain functions with magnetic resonance imaging systems has been done using fast pulse sequences. As described by P. A. Bandettini, E. C. Wong, R. S. Hinks, R. S. Tikofsky and J. S. Hyde, Time Course EPI of Human Brain Function During Task Activation, Magn. Reson. Med. 25, 390-397 (1992); J. Frahm et al., in “Dynamic MR Imaging of Human Brain Oxygenation During Rest and Photic Stimulation”, JMRI 1992:2:501-505; K. Kwong et al., in “Dynamic Magnetic Resonance Imaging of Human Brain Activity During Primary Sensory Stimulation”, Proc. Natl. Acad. Sci. USA Vol. 98, pp. 5675-5679, June 1992 Neurobiology; and S. Ogawa et al., “Intrinsic Signal Changes Accompanying Sensory Stimulation: Functional Brain Mapping Using MRI”, Proc. Nati. Acad. Sci. USA Vol. 89, pp. 5951-5955, June 1992 Neurobiology, these prior methods use a difference technique in which a series of image data sets are acquired with an EPI pulse sequence while a particular function is being performed by the patient, and a baseline image data set is acquired with no patient activity. The baseline data set is subtracted from the series of data sets to produce difference images that reveal those parts of the brain that were active during the performance of the function. These difference images may be displayed in sequence to provide a cine display of the activity-induced brain functions. As described by R. W. Cox et al., in “Real-Time Functional Magnetic Resonance Imaging,” Magn. Reson. Med. 33, 230-236 (1995), and C. S. Potter et al., in “THE NEUROSCOPE: An Interactive System for Real-Time Functional MRI Of The Brain”, Proceeding of the SMR, Vol. 2, 1994, these images may be displayed in real-time as the data is being acquired.

The difference in NMR signal level produced by regions of the brain that are active and those that are inactive has been found in prior work to be very small. The difference is believed to result from the increase in oxygen supply to active portions of the brain which decreases the susceptibility differential between vessels and surrounding tissues and is referred to in the art as the blood oxygenation level dependent (BOLD) contrast mechanism. This allows an increase in the phase coherence of spins and a resulting increase in NMR signal level. However, this difference in signal level is only 2 to 4 percent (at 1.5 Tesla) in pulse sequences that employ voxel sizes in the range of 40 mm³ to 50 mm³. In addition, this signal difference is masked by system noise, and artifacts caused by patient motion, brain pulsatility, blood flow and CSF flow.

In U.S. Pat. No. 5,603,322, entitled “Time Course MRI Imaging of Brain Functions”, a method is described for improving the quality of functional MRI (“FMRI”) images. This method correlates the NMR signal acquired over a period of time with a reference signal that corresponds with the expected signal. For example, if the subject is stimulated by turning a stimulus on and off in a pattern, those regions of the brain responsive to this stimulus will produce an NMR signal that correlates well with the pattern. Brain activity is thus measured by calculating the degree of correlation between the NMR signal at each voxel and the reference signal. As more NMR data is acquired, the correlation calculations become more time consuming, but the image quality also improves.

The classic model of the BOLD contrast mechanism which is the responsible for the fMRI signals indicates that scanner thermal noise and pulse sequence echo time (TE) are critical factors in image quality. As indicated by R. S. Menon, et al., “4 Tesla Gradient Recalled Echo Characteristics Of Photic Stimulation-Induced Signal Changes In The Human Primary Visual Cortex,” Magn. Reson. Med. 30:380-386 (1993), the prevailing model of fMRI contrast-to-noise ratio (CNR) is given as:

$$\frac{S ( fMRI )}{N} = \frac{{S}_{0}}{{N}_{T}} [ {ⅇ}^{- ( TE / {T2}^{* ′} )} - {ⅇ}^{- ( TE / {T2}^{*} )} ] , \qquad ( 1 )$$

where T2* and T2*′ are decay values in the absence of and in the presence of brain activation, and N_{T} is thermal noise of the scanner and is presumed to be white. S₀ is the acquired NMR signal, and it is different for every voxel. It can be determined in several possible ways including, for example, cross correlation of a voxel time course with a boxcar waveform as follows:

S₀=σ_{f}·σᵣ (2)

where σ_{f} is a vector formed from the points in an experimental pixel time course and σᵣ is a reference vector—for example, a boxcar waveform. In this example, N_{T} could be estimated similarly by cross correlation of a pixel time course that lies in free space and shows no evidence of ghosting, taking into account the numerical factor of 1/526 that converts thermal noise in free space to thermal noise in the presence of signal.

According to equation (1), S(fMRI) is zero when the pulse sequence used to acquire the NMR data has an echo time set at TE=0 or at TE=∞, and it has a maximum value when the echo time is set at TE=T2*. According to equation (1), use of pulse sequences with shorter TE values severely impacts the CNR. As a result, the usual practice in the art is to employ a single-shot EPI pulse sequence in which the center of k-space is acquired at TE=T2*. This results in longer scan times.

The same equation (1) indicates that the CNR of fMRI images may be increased by increasing the size of each voxel defined by the applied imaging gradients. Indeed, equation (1) indicates the CNR will vary inversely with the voxel volume. As a consequence, fMRI images are typically acquired with voxel sizes on the order of 3×3×5 mm³ (45 mm³) to produce images with sufficient CNR.

### SUMMARY OF THE INVENTION

The present invention is a method for producing fMRI images which are more responsive to brain activities and is based on the discovery that noise if fMRI imaging is dominated by fluctuations from the brain itself, rather than thermal noise N_{T} as expressed above in equation (1). It has been discovered that the CNR of fMRI images is enhanced if the voxel size of the acquired NMR data is matched with the size of the functionally active anatomic regions in the brain. More specifically, the imaging pulse sequence employed to acquire the fMRI data set acquires NMR data having cubic voxels with a size ranging from 1 to 8 mm³, which is a 10 to 50 times reduction in size over conventional fMRI acquisitions.

Another aspect of the invention is the discovery that an acquired fMRI signal is not maximum when the pulse sequence echo time TE is set to T2*. Instead, it has been discovered that CNR is substantially independent of the pulse sequence echo time TE over a range from one-third to three times T2*. This discovery is exploited by using an EPI pulse sequence which acquires only half of k-space, beginning with the acquisition of the center of k-space and with its TE set to one-third to one-half T2* such that the central views of k-space are acquired early in the free induction decay of the NMR signal.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims herein for interpreting the scope of the invention.

### BRIEF DESCRIPTION OF THE DRAWINGS

FIG. 1 is a pictorial representation of an MRI system which employs the present invention;

FIG. 2 is a graphic representation of a preferred pulse sequence used to acquire fMRI data according to the present invention;

FIGS. 3a-d are pictorial representations of various steps performed on the partial k-space data acquired with the pulse sequence of FIG. 2;

FIG. 4 is a pictorial representation of the reconstructed fMRI data acquired for one slice with the pulse sequence of FIG. 2; and

FIG. 5 is a graphic indication of the increased fMRI activation sensitivity when acquiring cubic voxels in the 1.0 to 3.0 mm size range.

### GENERAL DESCRIPTION OF THE INVENTION

Blood is a unique source of physiological contrast in MRI due to its oxygenation-sensitive paramagnetic characteristics. Deoxyhemoglobin contains paramagnetic iron, while oxyhemoglobin contains diamagnetic oxygen-bound iron. The partial pressure of oxygen in blood regulates the oxygen saturation of hemoglobin, as described empirically by the oxygen-hemoglobin dissociation curve.

It is well established that the oxygen saturation of hemoglobin affects the T₂ of whole blood. The susceptibility differential between the hemoglobin-containing erythrocyte and surrounding plasma creates microscopic field inhomogeneities. Irreversible spin dephasing is caused by exchange of protons across the erythrocyte membrane and/or diffusion of protons through the microscopic magnetic field gradients.

On a larger scale, it has been demonstrated that the paragamagnetic contribution of deoxyhemoglobin affects the susceptibility of whole blood, causing it to be less diamagnetic than surrounding tissue. Magnetic field inhomogeneities within and around each vessel are created by this susceptibility differential. A spin-echo signal is attenuated by spin dephasing due to diffusion of spins through field inhomogeneities, while a gradient-echo is additionally attenuated by dephasing due to static field inhomogeneities, independent of diffusion. Changes in T₂* are observed through the time course collection of images obtained with an echo-planar imaging (EPI) sequence which samples during the free induction decay. This sequence is referred to as gradient-echo EPI. The contrast provided in such fMRI images is based on blood oxygenated level dependent (BOLD) contrast.

It has become apparent to us that “noise” in an fMRI image contains a number of components other than the thermal noise component of the MRI scanner. These other noise components are produced by the brain as well as imperfections in the MRI scanner, and they include noise peaks of various magnitudes caused by patient respiration and the heart beat. It has also been discovered that an intense low frequency noise is produced in gray matter which arises from fluctuations in blood oxygenation through a spontaneous BOLD mechanism. It is apparent, therefore, that the prevailing model of fMRI CNR in equation (1) is not correct.

Based on our understanding, a better model of the fMRI CNR equation is as follows:

$$\frac{S ( fMRI )}{N} = {S}_{0} \frac{[ {ⅇ}^{- ( TE / {T2}^{* ′} )} - {ⅇ}^{- ( TE / {T2}^{*} )} ]}{{⟨ {\{ {N}_{B} [ {ⅇ}^{- ( TE / {T2}^{* ′} )} - {ⅇ}^{- ( TE / {T2}^{*} )} ] + {N}_{0} + {N}_{T} + {N}_{SC} \}}^{2} ⟩}^{1 / 2}} , \qquad ( 3 )$$

where: N_{B}=physiological noise of BOLD origin,

N₀=other physiological noise,

N_{T}=thermal noise, and

N_{SC}=scanner noise arising from system instabilities.

The noise term N_{SC} can be measured in a number of ways, but the teachings of the present invention have been found to be particularly useful. The N_{SC} term is measured using a phantom rather than a human subject. This phantom characteristically has edges of very high contrast such as occur when a piece of plastic is surrounded by water containing a paramagnetic relaxation agent. The measurement is performed by acquiring a time course of images. If a voxel contains a high contrast edge, the resulting pixel time course is very sensitive to scanner noise. A Fourier transform of this pixel time course reveals the frequencies that characterize scanner instability. Scanner instability arises, for example, from fluctuations of the magnetic environment (vehicles or elevators), gradient amplifier power supplies, shim current power supplies, mechanical vibrations, and RF instabilities. The quality of the measurements of N_{SC} depends on the temporal and spatial resolution of the acquired data. Just as the methods of this invention enhance spatial and temporal resolution for human brain, they also similarly enhance measurement of scanner noise and instabilities.

The four noise terms in equation (3) are functions of time, and N_{B}, N₀ and possibly N_{SC} are pixelwise dependent. Brackets in the denominator of equation (3) denote time course averaging, noting that all of these various sources of noise may not be independent. Since the noise is structured, analysis of equation (3) must be carried out in the frequency domain. For example, a boxcar reference vector σᵣ has components at the first, third, fifth . . . harmonics of the repetition frequency. Because of the spectral dependence of physiological noise, the noise at these various harmonics progressively decreases as the harmonic number increases and also as the epoch duration decreases. This model predicts that if the physiological noise of BOLD origin N_{B} term dominates the other noise terms, S(fMRI)/N is independent of TE. It also predicts that in the absence of partial voluming of the activated volume, the CNR should be independent of voxel volume. In addition, the noise should be proportional to the percent enhancement for all activated voxels, which leads to the further observation that even though the noise varies from pixel to pixel, the CNR should be constant from pixel to pixel.

This discovery is exploited in two ways. First, because the CNR is substantially independent of voxel size, the pulse sequence used to acquire fMRI data may be designed to acquire NMR signals from voxels that are matched in size and shape to the functionally distinct anatomic regions in the brain. And second, because the CNR is substantially independent of TE, a shorter pulse sequence echo time may be used with a consequent reduction in scan time.

If the voxel size is matched to the functionally distinct anatomic regions, the fMRI image more accurately resolves brain activity. When the voxel is too large, a much smaller region of the brain which is active produces an increased NMR signal which is “diluted” by the surrounding inactive regions within the same voxel. Thus, even though the active region registers maximum activity, this does not result in a maximum possible increase in NMR signal intensity. This “partial volume” effect is prevalent in current fMRI procedures and results in decreased dynamic signal range in addition to reduced spatial resolution.

It is believed that the BOLD signal which indicates brain function has as one of its origins a vascular layer in gray matter which has a thickness of 0.8 mm. This layer has the highest capillary density and thus the highest metabolic demand. As discussed by H. M. Duvernoy et al., “Cortical Blood Vessels Of The Human Brain,” Brain Research Bulletin 7:519-579 (1981), intracortical penetrating veins may be classified into five groups. Group 3 penetrates to neuronal layer IV, group 4 to neuronal layer V, and group 5 (so-called principal veins) through to white matter. It is believed that groups 3, 4 and 5 participate in BOLD contrast. These veins are of progressively greater diameter, each fed by numerous branching capillaries in the gray matter vascular layer. These have diameters of 120 to 125 μ, 65 μ, and 45 μ for groups 5, 4 and 3, respectively. Penetrating arteries surround the principal veins as well as veins in groups 3 and 4, and these structures of arterial rings surrounding single veins are referred to as “venous units.” The number of penetrating arteries is much greater than the number of veins, and the larger the diameter of the veins the greater the number of penetrating arteries that surround it. The tangential area that is drained by veins in groups 3 and 4 ranges from 0.75 to 1 mm in diameter, while the area drained by principal veins ranges from 1 to 4 mm.

From an anatomic sense, it appears that the ideal voxel size for fMRI based on the BOLD contrast mechanism is a 1 mm cubic voxel. Tests have been done using 1-2 mm cubic voxels with great success. Results from these experiments lead to the conclusion that a 1.5×1.5×1.5 mm³ voxel size is preferable from the perspective of optimum tradeoff between partial volume effects and contrast-to-noise ratio.

### DESCRIPTION OF THE PREFERRED EMBODIMENT

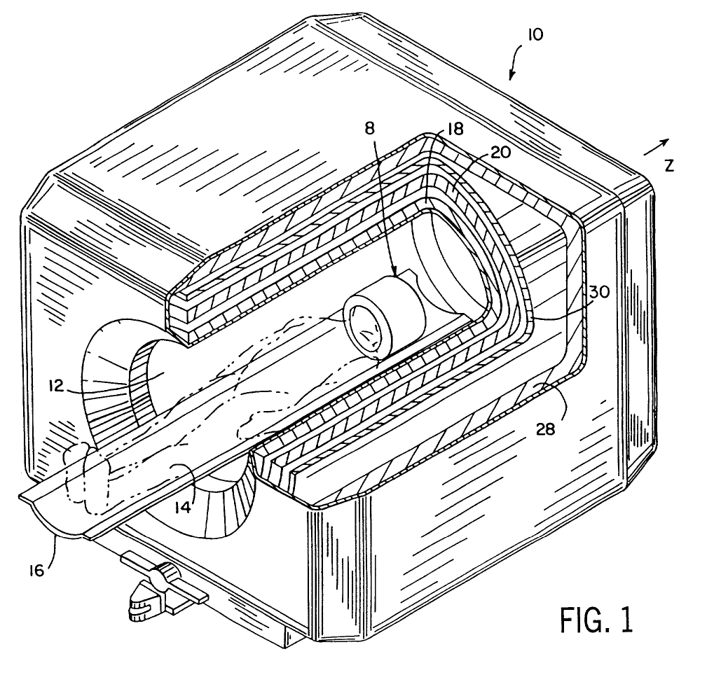

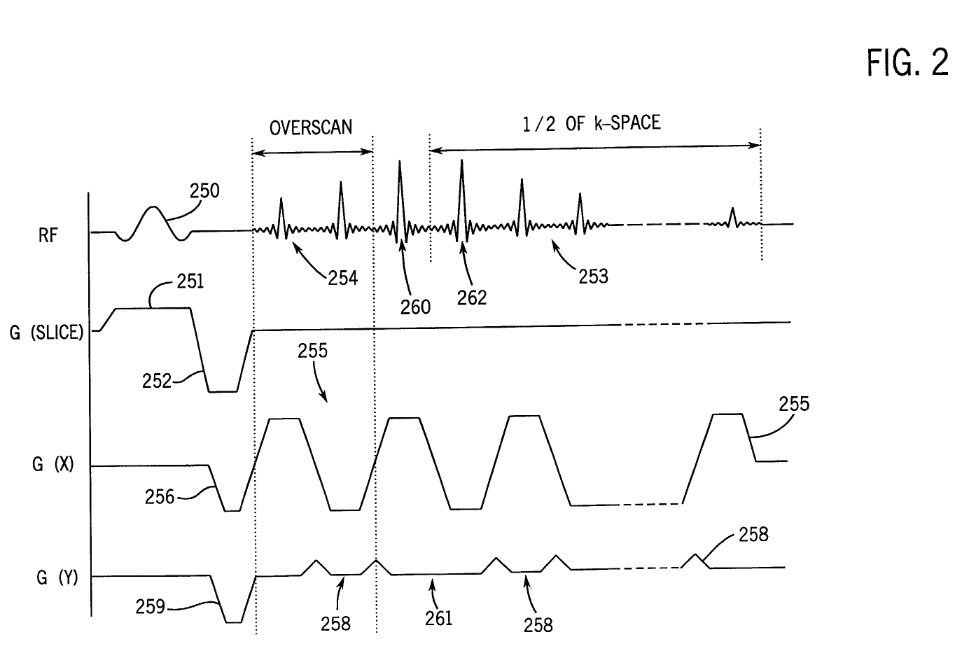

Referring to FIG. 1, an MRI magnet assembly 10 has a cylindrical bore tube 12 extending along a z-axis 13 for receiving a supine patient 14 supported on a table 16. The table 16 may move in and out of the bore tube 12 so as to position the patient 14 along the z-axis 13 within the volume of the bore tube 12.

Coaxially surrounding the bore tube 12 is a whole-body RF coil 18 for exciting the spins of the patient 14 into resonance, as has been described. Whole-body gradient coils 20 surround both the bore tube 12 and the RF coil 18 and are also coaxial with the z-axis 13, to provide x, y and z gradient fields Gₓ, G_{y} and G_{z} as required for MRI imaging. The gradient coils 20 are driven by gradient amplifiers (not shown). The polarizing magnetic field B₀, aligned with the z-axis 13 is generated by a superconducting magnet coil 28 coaxial with but outside the bore tube 12, the RF coil 18 and the gradient coils 20. The superconducting magnet coil 28 has no external power supply but operates on an initial current which continues unabated in the zero resistivity windings of the superconducting magnet coil 28.

Interposed between the superconducting magnet coil 28 and the gradient coil 20 is a set of shim coils 30 which are used to correct the homogeneity of the polarizing field B₀ as is understood in the art. A set of mechanical linkages and insulators (not shown) rigidly connect each of these coils 18, 20, 28 and 30 together to the bore tube 12 so as to resist relative motions generated by the interaction of their various electromagnetic fields.

When a local coil assembly 8 is used in a general purpose system such as that described above, the whole-body gradient coils 20 and whole-body RF coil 18 are disconnected. The local coil assembly 8 is connected to the x, y and z gradient amplifiers (not shown) on the NMR system and it is connected to the system's transceiver through a transmit/receive switch. The preferred embodiment employs a 3 Tesla MRI system manufactured by Bruker Analytische MeBtechnik GmbH and sold under the trademark BIOSPEC 30/60.

Because the gradient fields are switched at a very high speed when an EPI sequence is used to practice the preferred embodiment of the invention, local gradient coils are employed in place of the whole-body gradient coils 139. These local gradient coils are designed for the head and are in close proximity thereto. This enables the inductance of the local gradient coils to be reduced and the gradient switching rates increased as required for the EPI pulse sequence. The local gradient coil assembly 8 also includes a local brain RF coil. In the preferred embodiment, it is a 16 element bandpass endcapped birdcage coil. This brain RF coil is designed to couple very efficiently to the brain of the subject and less efficiently to the lower part of the head. This results in improved brain image quality compared with larger general purpose head coils that couple uniformly to the entire head as well as the neck. An RF shield surrounds the local brain coil and interior to the local gradient coil. This shield isolates RF radiation from the local gradient coil. The shield is designed to avoid perturbation of time varying gradient fields. For a description of these local gradient coils and the RF coil which is incorporated herein by reference, reference is made to U.S. Pat. No. 5,372,137 filed on Jan. 19, 1993 and entitled “NMR Local Coil For Brain Imaging”.

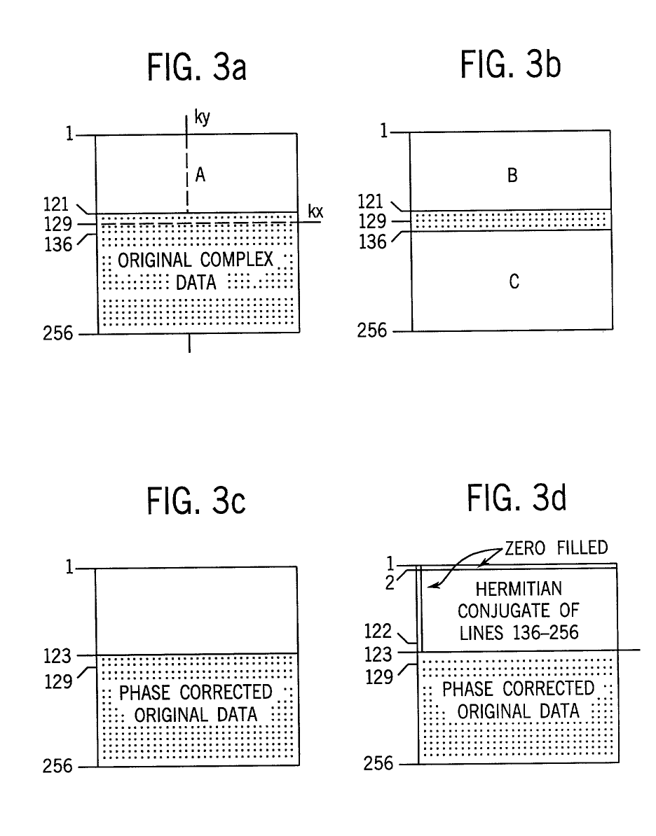

The EPI pulse sequence employed in the preferred embodiment of the invention is illustrated in FIG. 3. A 90° RF excitation pulse 250 is applied in the presence of a G_{z} slice select gradient pulse 251 to produce transverse magnetization in a slice through the brain ranging from 1 to 2 mm thick. The excited spins are rephased by a negative lobe 252 on the slice select gradient G_{z} and then a time interval elapses before the readout sequence begins. A total of 128 separate NMR echo signals (or “views”), indicated generally at 253, is acquired during the EPI pulse sequence along with 8 overscan views 254. Each NMR echo signal 253 is a different view which is separately phase encoded to sample a line in k-space.

The NMR echo signals 253 are gradient recalled echo's produced by the application of an oscillating Gₓ readout gradient field 255. The readout sequence is started with a negative readout gradient lobe 256 and the echo signals 253 are produced as the readout gradient oscillates between positive and negative values. A total of 256 samples are taken of each NMR echo signal 253 during each readout gradient pulse 255. The successive NMR echo signals 253 are separately phase encoded by a series of G_{y} phase encoding gradient pulses (or “blips”) 258. The first pulse is a negative lobe 259 that occurs before the echo signals are acquired to encode the first overscan view at k_{y}=−8. Its area is such that after the eight overscan views are acquired the center of k_{y} space is reached and a first central view 260 is acquired. One phase encoding pulse is deleted at 261 such that a second central view 262 is acquired with an opposite polarity readout gradient 255. Subsequent phase encoding pulses 258 occur as the readout gradient pulses 255 switch polarity, and they step the phase encoding monotonically upward through k_{y} space (k_{y}=1−128). These 128 views that sample one-half of k-space are thus acquired in a centric view order, that is, a view order in which k-space is sampled beginning at the center of k-space and extending toward the periphery of k-space.

The two central views 260 and 262 are used for horizontal (x-axis in k-space) phase and frequency-offset correction. One advantage of the preferred pulse sequence is that these two views are acquired at minimal delay after the 90° pulse 250 and exhibit high SNR. As described below, the eight overscan views 254 are needed to produce the phase map that is necessary to center the central echo on the central pixel, which is required to fill the empty views of k-space (k_{y}=−9 to −128).

As is well known to those skilled in the art, fast Fourier transforms are usually structured in powers of two, resulting in matrix sizes of 64×64, 128×128 or 256×256. So called radix fast Fourier transforms have also been used. An example is a matrix of 192×192, noting that 192=2 to the sixth power times 3. Other numerical combinations are possible. The human brain in all three projections, sagital, axial and coronal, can be covered using 16×16 cm field of view without aliasing. Often 20×20 cm is used. Axial slices at the top of the head can be covered with a field-of-view of 12.8×12.8 cm, which is particularly convenient using a matrix size of 128×128. The resulting pixel size is 1 mm. The field-of-view can readily be adjusted in small increments, for example 19.2×19.2 cm. In this case a matrix size of 192×192 also results in a pixel size of 1 mm. It will be appreciated that many combinations of matrix size and field-of-view are possible within the scope of the invention.

The order of data acquisition in this preferred pulse sequence permits the echo time TE to be as short as possible. Echo times and readout times as a function of spatial resolution are shown in Table 1 for full and half k-space EPI using the scanner parameters given above.

TABLE 1
Echo and Readout Times (ms)

| | |
|---|---|
| TE | Readout Time |

| K-space | Full | Half | Full | Half |
|---|---|---|---|---|
| matrix size | k-space | k-space | k-space | k-space |
| 64 × 64 | 23.0 | 9.2 | 37.4 | 23.6 |
| 128 × 128 | 66.4 | 12.6 | 123.8 | 70.1 |
| 192 × 192 | 134.4 | 16.1 | 259.4 | 141.1 |
| 256 × 256 | 226.0 | 19.5 | 444.1 | 236.7 |

As can be seen from Table 1, the echo time for half k-space acquisition is about one-half of T₂* for a 256×256 matrix, and the readout time exceeds T₂* by a factor of 5 or 6 for a full k-space acquisition of the same matrix size. For fMRI, it is estimated that the use of a TE of 20 ms with a T₂* of 40 ms reduces FMRI contrast relative to use of a TE of 40 ms by 15%, which is acceptable. However, a calculation of fMRI contrast for full k-space 256×256 acquisition assuming T₂* =40 ms and TE=226.9 ms shows that the contrast drops by a factor of 20 relative to the contrast when TE=40 ms.

It follows that the total acquisition time (TACQ) should be as short as possible. The value of TACQ can be calculated using the following expression

TACQ=(TSAMP+XRES+2RAMPTIME)[(YRES/2)+OVS+1] (4)

where the matrix size is XRES by YRES, the number of overscan lines (OVS) is typically eight, and the ramp time (RAMPTIME) is typically 100 microseconds. The sampling time, TSAMP, is given by the expression

TSAMP=1/BW=2 π(γFOV Gₓ). (5)

The bandwidth (BW) is typically 125 kHz, γ is the gyromagnetic ratio of protons, the field of view (FOV) is the object size in the X dimension that is to be imaged, typically 16 cm, and Gₓ is the gradient strength in the X dimension.

Referring to equation 4, the use of half k-space acquisition is indicated by the term YRES/2. The factor of ½ substantially reduces TACQ. For a given resolution value, reduced T2* decay occurs since TACQ is reduced. Conversely, equation 4 shows that that if TACQ corresponds to an acceptable amount of T2* decay during an image acquisition, the resolution can be increased when using half k-space acquisition. TSAMP must also be as small as possible in order to reduce TACQ, which requires a large value of BW. It follows from Equation (5) that Gₓ should be large since the field-of-view, FOV, is determined by the dimensions of the brain itself. Large Gₓ values with short ramp times are achieved more readily with local gradient coils than with body gradient coils. Previously, following Equation (1), a large value of BW degrades the signal-to-noise ratio because of increase in thermal noise, N_{T}. Equation (3) teaches that this is no longer true if the thermal noise, N_{T}, is substantially smaller than the other terms in the denominator of this equation, N_{B}, N_{T}, N_{SC} that contribute to the noise.

A central issue in single-shot high-resolution GR-EPI arises in the trade-off between the number of lines of k-space that must be acquired and decay of signal intensity because of T₂*. Use of partial k-space acquisition reduces the number of views that must be acquired for a given matrix size by approximately a factor of two. This in turn permits the use of a shorter echo time TE. The reduction in TE for half k-space acquisition with centric view ordering becomes increasingly significant as the resolution increases (see Table 1) because the acquisition of the center of k-space progressively drops in intensity for full k-space acquisition.

It is a teaching of the present invention that the scan parameters should be set such that the EPI pulse sequence samples k-space along each of the k-space axes kₓ, and k_{y} in substantially equal increments. These increments along with the slice thickness are selected such that the three-dimensional image reconstructed from the sampled k-space data set has cubic voxels of a size ranging from 1.0 mm to 2.0 mm. These voxel sizes are believed to optimally match the anatomic structures in the brain which produce changes in the fMRI signal intensity during brain activity.

A complete scan is performed in which the EPI pulse sequence is repeated 128 times for each slice to acquire time course NMR data for 128 slice images. The EPI pulse sequences are spaced apart in 2 to 3 second intervals such that the entire time course acquisition spans a 4 to 6 minute time period. In a typical scan 10 contiguous 1.5 mm axial slices are acquired through the subject's brain to acquire fMRI data from 1.5 mm cubic voxels throughout a 16×16×1.5 cm slab. During the scan the subject is asked to perform a specific function in a predetermined pattern, or a stimulus is applied to the subject in a predetermined pattern. For example, the subject may be instructed to touch each finger to his thumb in a sequential, self-paced, and repetitive manner, or the subject may be subjected to a sensory stimulus such as a smell or visual pattern in a periodic manner. More than one such experiment may be conducted during the scan by varying the repetition rate, phase, or frequency, of the applied stimulus or performed function so that they can be discriminated on the basis of the frequency difference.

At the completion of the scan a series of partial k-space data sets are stored for each slice location. Each partial k-space data set is completed using a method similar to that described by D. E. Purdy, “A Fourier Transform Method Of Obtaining High Resolution Phase Maps For Half-Fourier Imaging,” Proc. SMRM, 7^{th} Annual Meeting, San Francisco, 1998, pg. 968.

FIG. 3a is a diagram of k-space in which views actually acquired are indicated by the shaded area. In addition to acquisition of half k-space views 129-256, N overscan lines are acquired adjacent to line 129. In the preferred embodiment N is set to 8, although the software enables other values to be set. Acquisition begins with line 121 and proceeds to line 256.

According to the symmetries of the FT, if the raw data have a symmetrical real part (I) and an asymmetrical imaginary part (Q), then the image is purely real. The first step in reconstruction is to center the data on line 129 of k-space such that I and Q have the requisite symmetries. The reduced I and Q matrices are formed from the lines of k-space shown in FIG. 3b, inserting zeroes in spaces B and C. These data are Fourier-transformed to produce 256×256 real and imaginary images. From these images, a pixel-by-pixel phase map (arc tan(I_{M}/Q_{M})), where I_{M} and Q_{M} refer to the image real and imaginary intensities, is constructed and saved. This phase map has dimensions of 256×256, but actually has 256 resolution only in the x direction. It is smoothes in the y direction as would be expected for 2N resolution.

The original data set (FIG. 3a) is transformed to image space and the phase map is used to correct the values such that all information resides in I_{M} and no intensity is left in Q_{M} except for small discrepancies between the actual y axis image resolution and the y axis smoothed phase map. The phase-corrected image is then brought back to k-space by inverse FT (FIG. 3c). The data are now centered on line 129. With the data centered and phase corrected, the top part of k-space is filled by the Hermitian conjugate of the lower part according to equation (5) and as shown in FIG. 3d:

raw(−kx, −ky)=raw*(kx, ky) (6)

Note that only lines 2-122 are filled. No data exist to fill line 1, and it is set to zero. It is also necessary to zero-fill one-half of a vertical column, as indicated in FIG. 3d. Original phase-corrected lines for 123-128 and two Hermitian conjugate lines for 121 and 122 was determined empirically and is a trade-off between SNR and artifacts. Finally, the data of FIG. 3d are transformed to image space by performing a two-dimensional Fourier transformation thereof. The final image is produced by forming a magnitude image [I_{M}²+Q_{M}²]^{½}.

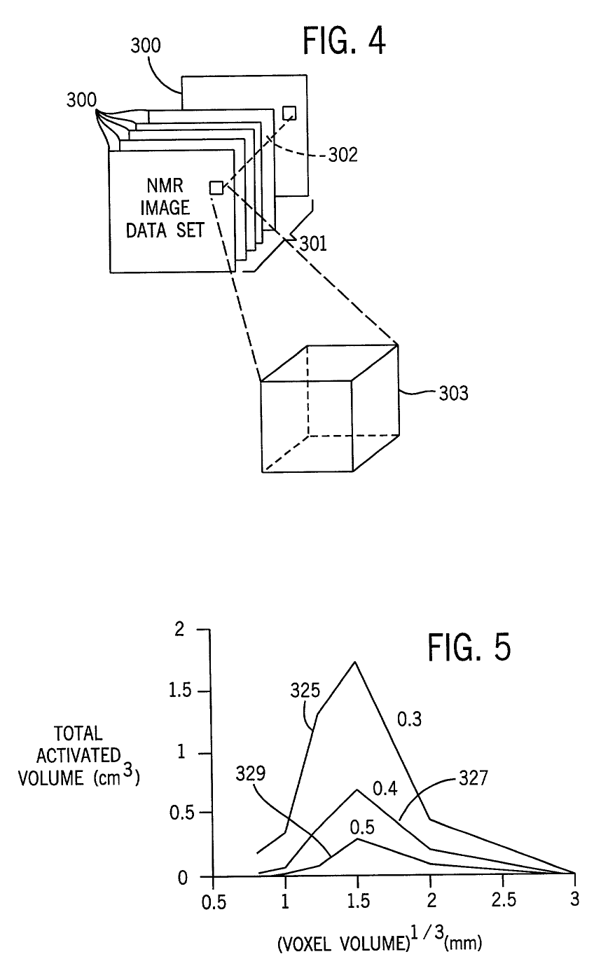

Referring particularly to FIG. 4, the resulting fMRI data set for each image slice is organized as a set of 256×256 element 2D arrays 300 in which each element stores the magnitude of the NMR signal from one voxel 303 in the scanned slice. Each array 300 can be used to directly produce a 256×256 pixel anatomical image of the brain slice for output to video display. While each array 300 is a “snap shot” of the brain slice at a particular time during the time course study, the NMR image data set may also be viewed as a single 256×256×128 3D array 301 in which the third dimension is time.

The time course NMR image data for one voxel 303 in the array 301 is referred to herein as a time course voxel vector. One such 128 element vector is illustrated in FIG. 4 by the dashed line 302. Each time course voxel vector 302 indicates the magnitude of the NMR signal at a cubic voxel in the image slice over the time course study. The resulting time domain voxel graph reveals very clearly variations in the activity of the brain in the region of the voxel 303. Regions which are responsive to a sensor stimulus, for example, can be located by identifying time domain voxel graphs which vary at the same repetition rate as the applied stimulus.

An fMRI image of the brain may be produced from the fMRI data set in a number of ways. As described in U.S. Pat. No. 5,603,322 in the preferred embodiment the fMRI image is produced by correlating each time course voxel vector 302 with a reference vector which depicts the activation or stimulation pattern that is producing the brain activity. A correlation number from 0 to 1.0 is produced for each 1.5 cm voxel in the acquired slab and this correlation number may be used to modulate the intensity or color of the corresponding pixel in the display image.

Referring particularly to FIG. 5, the sensitivity of the fMRI method has been measured as a function of cubic voxel size. A 6 mm thick slab of fMRI data was acquired from activated tissue in the motor cortex of a subject. Acquisitions were done with cubic voxels of 0.8, 1.0, 1.3, 1.5, 2.0 and 3.0 mm using the pulse sequence and fMRI post processing method described above. The number of voxels in the 6 mm slab that yielded correlation coefficients greater than 0.3, 0.4 and 0.5 was calculated for each acquisition. The number of activated voxels times the volume of each voxel provides an indication of the total volume of tissue which is found to be activated.

As shown in FIG. 5, the greatest fMRI sensitivity at all three correlation coefficient thresholds is achieved with a voxel size of 1.5 mm on each side. The highest activated volume is achieved when the correlation coefficient is the lowest (i.e. 0.3) as indicated by curve 325, the next highest is achieved when the correlation coefficient threshold is set to 0.4 as indicated by curve 327, and the lowest is achieved when the correlation coefficient must exceed 0.5 as indicated by curve 329. The increase in sensitivity in the 1.0 to 2.0 mm cubic voxel size range is unmistakable and quite surprising.

It should be apparent that variations are possible from the preferred embodiment without departing from the spirit or scope of the invention. While a cubic voxel is believed to provide optimal results, good results can also be achieved if one or two of the voxel dimensions vary slightly from a perfect cube.

It should be apparent to those skilled in the art and practice of functional magnetic resonance imaging that this application is not limited to functional neuroimaging based on BOLD contrast induced by task performance. Other types of BOLD contrast exist including: changes in blood oxygenation that are secondary to respiration, changes in blood oxygenation that are secondary to cardiac activity, and changes in blood oxygenation and flow that are in response to hypercapnia. Studies based on these changes can rightly be termed “functional magnetic resonance imaging” and are included in the scope of this invention. In addition, BOLD contrast is associated with simultaneous changes in bold volume and blood flow. The method and theory of the present application can be translated in a straightforward manner to strategies based on contrast arising from such changes in blood flow and volume. Physiological fluctuations are no doubt present in all imaging studies in the living human subject and would be apparent in a time course of echo planar images. The scope of the invention includes all functional magnetic resonance studies wherein a time course of images is acquired and wherein the limiting noise source arises from fluctuations of physiology as well as from movements in the human body itself.

1. A method for producing a functional magnetic resonance image (fMRI) of a subject's brain, the steps comprising:
a) operating a magnetic resonance imaging (MRI) system to perform a series of pulse sequences that acquire a series of NMR k-space data arrays over a period of time, during which the subject's brain is caused to function in a preselected temporal pattern;
b) producing a time course NMR image data set of time domain voxel vectors from the series of k-space arrays, in which each time domain voxel vector indicates the NMR signal during said period of time from a substantially cubic region of the brain having a size from 1.0 to 8.0 mm³;
c) producing an image which indicates the amount of brain activity in each of said cubic regions.

2. The method as recited in claim 1 in which each pulse sequence is performed by the MRI system in step a) by:
i) producing an RF excitation pulse in the presence of a slice select magnetic field gradient aligned along a first k-space axis to produce transverse magnetization in a slice perpendicular to said first k-space axis with a thickness of from 1.0 to 2.0 mm;
ii) producing a series of phase encoding magnetic field gradients directed along a second k-space axis in the plane of the slice;
iii) producing a series of readout magnetic field gradients directed along a third k-space axis perpendicular to the second k-space axis, the series of readout gradients being produced concurrently with the series of phase encoding gradients;
iv) acquiring a series of NMR signals in the presence of the readout gradient to produce k-space data which samples k-space as a series of views in centric view order.

3. The method as recited in claim 2 in which the pulse sequence is performed such that a part of k-space is sampled by the acquired NMR signals and step a) includes:
v) calculating k-space samples for the unacquired part of k-space to form a complete k-space data array.

4. The method as recited in claim 3 in which the acquired part of k-space is substantially one-half of k-space.

5. The method as recited in claim 1 in which step a) acquires a series of NMR k-space data arrays from a plurality of substantially contiguous slices through the subject's brain.

6. The method as recited in claim 5 in which the period of time is from four to six minutes.

7. The method as recited in claim 2 in which the pulse sequence is a multislice, partial k-space, gradient recalled echo planar imaging pulse sequence.

8. The method as recited in claim 2 in which steps i) and iv) are performed using a local RF coil configured for imaging the human brain.

9. The method as recited in claim 2 in which steps ii) and iii) are performed using a local gradient coil configured for imaging the human brain.

10. The method as recited in claim 1 in which the pulse sequence is selected to detect changes in brain activity produced by the BOLD contrast mechanism.
